(19) United States
(12) Patent Application Publication
(10) Pub. No.: US 2026/0137720 A1
(43) Pub. Date: May 21, 2026

(54) THERAPIES FOR NEUROMUSCULOSKELETAL INJURIES AND LIMB TRANSPLANTATION

(71) Applicant: The Government of the United States, as Represented by the Director of the Defense Health Agency, Ft. Detrick, MD (US)

(72) Inventor: Shashikumar Krishnaji SALGAR, University Place, WA (US)

(21) Appl. No.: 19/303,268

(22) Filed: Aug. 18, 2025

Related U.S. Application Data
(60) Provisional application No. 63/684,840, filed on Aug. 19, 2024.

(51) Int. Cl. A61K 35/28; A61K 35/16; A61K 38/19; A61K 38/20; A61N 7/00; A61P 25/02; C12N 5/0775

(52) U.S. Cl. CPC A61K 35/28; A61K 35/16; A61K 38/193; A61K 38/2066; A61N 7/00; A61P 25/02; C12N 5/0663; A61N 2007/0026; C12N 2510/00

(57) ABSTRACT

The present invention relates to the use of mesenchymal stem cells to reduce inflammation and immune responses for neuromuscular injuries and limb transplants. A particular preferred embodiment relates to use of mesenchymal stem cells that are modified to express interleukin-10 in limb transplantation surgery. In particular embodiments, the present invention also provides a method of improving nerve and muscle regeneration and functional outcome for limb transplantation in a mammalian limb transplant recipient by administering to the limb transplant recipient a therapeutic amount of mesenchymal stem cells.

### CROSS-REFERENCE TO RELATED APPLICATIONS

This application is a United States Nonprovisional Application, which claims the benefit of U.S. provisional application Ser. No. 63/684,840, filed 19 Aug. 2024. The entire contents of the aforementioned application is hereby incorporated by reference as if fully set forth herein.

### GOVERNMENT FUNDING SUPPORT

This invention was made with government support under grant no. DM190329: CDMRPL-19-0-DM190329, awarded by CDMRP, DoD. The government has certain rights in the invention.

### REFERENCE TO ELECTRONIC SEQUENCE LISTING

The application contains a Sequence Listing which has been submitted electronically in .XML format and is hereby incorporated by reference in its entirety. Said .XML copy, created on Dec. 30, 2025, is named “10457-043US1_seq.xml” and is 4,405 bytes in size. The sequence listing contained in this .XML file is part of the specification and is hereby incorporated by reference herein in its entirety.

### BACKGROUND

### 1. Field of the Invention

The invention relates to the general field of medicine, and specifically to the use of mesenchymal stem cells in order to reduce inflammation and immune responses for neuromuscular injuries and limb transplants. A particular preferred embodiment relates to use of mesenchymal stem cells that are modified to express interleukin-10 in limb transplantation surgery.

### 2. Background of the Invention

Severe trauma, whether in battlefield or civilian circumstances such as labor or traffic accidents, can lead to limb amputations such as arm, hand, finger, leg, and foot, or other body parts due to severe neuromusculoskeletal injury. Transplantation is increasingly needed to improve the quality of life in military and civilian populations upon serious injuries of this kind in order to reduce long-term disability, and allow victims of such types of severe trauma to return to a more normal life and work.

Transplantation is a surgical procedure to transfer or attach a functionally active body part/organ from a recent dead person including a brain-dead donor to a recipient patient who is in need of that body part/organ. Limb transplant involves transplantation of multiple tissues as a single functional unit, termed a vascularized composite allotransplantation (VCA), preferably between well-matched donor-recipient pairs. The surgery involves connection of the bone or bones, tendons, arteries, veins, nerves, muscles and skin of the limb.

For success of the transplant, the transplant recipient must be treated with immunosuppressive drugs to minimize rejection of the transplanted tissues. Immunosuppressive medication regimens and intensive physical therapy improves chances of gaining function of the transplanted limb. However, these immunosuppressive drug regimens have whole body side effects that can eventually result in rejection of the limb or increased risk of infections and some cancers. For example, an immunocompromized patient on immunosuppressive drug therapy can have increased susceptibility to opportunistic invention and decreased cancer immunosurveillance. In any case, transplant rejection remains a constant threat for the life of the transplant, is not generally considered reversible or readily treatable, and is a major cause of transplant failure.

### SUMMARY OF THE INVENTION

Thus, there exists a need in the art for methods to improve healing, tissue regeneration, and successful functional outcomes, as well as to discourage rejection of transplanted limbs. In particular embodiments, the present invention relates to a method of improving nerve and muscle regeneration and functional outcome for limb transplantation in a mammalian limb transplant recipient in need thereof, comprising administering to the limb transplant recipient a therapeutic amount of mesenchymal stem cells.

In certain embodiments, the mammalian limb transplant recipient is a human. The limb can be selected from a digit, a hand, an arm, a foot, and a leg. In a preferred embodiment, the mesenchymal stem cells are genetically modified to express interleukin-10.

In certain embodiments of the invention, inflammation is reduced in the transplanted limb, graft versus host disease is reduced in the limb transplant recipient and/or the functional outcome of the limb transplant is improved.

In certain embodiments, the methods of the invention further comprise administering concurrent or sequential shock wave therapy to the limb transplant recipient and/or further comprise administering G-CSF and/or platelet-rich plasma therapy and/or shockwave therapy to the limb transplant or peripheral nerve injury recipient.

In additional embodiments, the invention relates to a genetically modified mesenchymal stem cell that expresses interleukin-10 and to a pharmaceutical composition comprising a pharmaceutically acceptable carrier and the above genetically modified mesenchymal stem cell.

### BRIEF DESCRIPTION OF THE DRAWINGS

Certain embodiments are illustrated by way of example, and not by way of limitation in the figures of the accompanying drawings.

FIG. 1 is a vector map, and provides SEQ ID NO:1.

### DETAILED DESCRIPTION OF EMBODIMENTS OF THE INVENTION

### 1. Overview

The present invention relates to a method for reducing inflammation and immune responses triggered by transplanted tissue during limb transplantation. The method involves using autologous mesenchymal stem cells alone and that are modified to express interleukin-10 (IL-10).

### 2. Definitions

Unless defined otherwise, all technical and scientific terms used herein have the same meaning as commonly understood by one of ordinary skill in the art. Although various methods and materials similar or equivalent to those described herein can be used in the practice or testing of the present invention, suitable methods and materials are described below. However, the skilled artisan understands that the methods and materials used and described are examples and may not be the only ones suitable for use in the invention. Moreover, as measurements are subject to inherent variability, any temperature, weight, volume, time interval, pH, salinity, molarity or molality, range, concentration and any other measurements, quantities or numerical expressions given herein are intended to be approximate and not exact or critical figures unless expressly stated to the contrary.

In the foregoing specification, the invention has been described with reference to specific embodiments thereof. It will, however, be evident that various modifications and changes may be made thereto without departing from the broader spirit and scope of the invention. The specification and drawings are, accordingly, to be regarded in an illustrative rather than a restrictive sense. Throughout this specification and the claims, unless the context requires otherwise, the word “comprise” and its variations, such as “comprises” and “comprising.” will be understood to imply the inclusion of a stated item, element or step or group of items, elements or steps but not the exclusion of any other item, element or step or group of items, elements or steps. Furthermore, the indefinite article “a” or “an” is meant to indicate one or more of the item, element or step modified by the article.

As used herein, the term “about” means plus or minus 20 percent of the recited value, so that, for example, “about 0.125” means 0.125±0.025, and “about 1.0” means 1.0±0.2. Notwithstanding that the numerical ranges and parameters setting forth the broad scope of the invention are approximations, the numerical values set forth in specific non-limiting examples are reported as precisely as possible. Any numerical value, however, inherently contains certain errors necessarily resulting from the standard deviation found in their respective testing measurements at the time of this writing. Furthermore, unless otherwise clear from the context, a numerical value presented herein has an implied precision given by the least significant digit. Moreover, all ranges disclosed herein are to be understood to encompass any and all sub-ranges subsumed therein. For example, a range of “less than 10” can include any and all sub-ranges between (and including) the minimum value of zero and the maximum value of 10, that is, any and all sub-ranges having a minimum value of equal to or greater than zero and a maximum value of equal to or less than 10, e.g., 1 to 4.

As used herein, the term “mesenchymal stem cells (MSCs)” (also known as mesenchymal stromal cells) are multipotent stromal cells that can differentiate into cells types such as osteoblasts, chondrocytes, myocytes and adipocytes. MSCs can be found throughout the body and MSC from any source within the body are contemplated for use with the invention, but MSCs used in examples below were derived from bone marrow. Cultured MSCs generally express CD73, CD90 and CD105 surface markers, but lack CD11b, CD14, CD19, CD34, CD45, CD79a, and HLA-DR surface markers. Bone marrow-derived MSCs do not express CD34.

As used herein, the term “interleukin-10 (IL-10)” (also known as human cytokine synthesis inhibitory factor is an anti-inflammatory cytokine encoded by the IL-10 gene on chromosome 1 in humans and primarily produced by monocytes. T helper cells, mast cells, and others.

As used herein, the term “nerve regeneration” generally refers to the processes of nerve regrowth after injury or bisection, including elongation of existing axons, sprouting and growth of new axons from neural cell soma, remyelination, plasticity among surviving connections, and functional recovery.

As used herein, the term “muscle regeneration” generally refers to a multi-step process that ends in a functional repair.

As used herein, the term “functional outcome” in the context of a transplanted limb refers to a regaining the natural function (sensory and motor) of the limb, for example the cutaneous pain reaction test a test that is performed by artificially stimulating the nerve boundaries in the rat paw by pinching with a manual forceps or electrostimulation and observing foot withdrawal and/or vocalization as sensory function response to stimulation as we have described previously. Motor function is determined by walking track analysis based on foot prints and measuring toe to toe and toe to heal spread distances and converting them into a Sciatic Function Index (SFI) which is a standard method of documenting motor function in rats as we have described previously. Motor function can be determined by Cat-Walk system a sophisticated electronic system that measures motor function response based on walking track/gait analysis electronically. Improved functional outcome refers to increases in levels of sensory and/or motor function and control of the transplanted limb compared to that which occurs in the absence of the inventive treatments.

As used herein, the term “mammalian limb transplant recipient” refers to any mammal that has undergone a transplant to receive a limb. A mammalian limb transplant recipient in need thereof refers to such a mammal that has, has had, may have, is suspected of having, or is susceptible to rejection of the transplanted limb.

As used herein, the term “limb” refers to a digit (i.e., a finger, thumb or toe), a hand, an arm, a foot, and a leg.

As used herein, the term “genetically modified” refers to a cell or cells that have been modified to contain and express a gene not normally expresses in that cell.

As used herein, the term “G-CSF” refers to Granulocyte-Colony Stimulating Factor.

As used herein, “administering” and its cognates refers to introducing an agent to a subject, and can be performed using any of the various methods or delivery systems for administering agents, pharmaceutical compositions or delivering gene vectors known to those skilled in the art. Modes of administering include, but are not limited to oral administration or intravenous, subcutaneous, intramuscular or intraperitoneal injections, rectal administration by way of suppositories or enema, or local administration directly into or onto a target tissue (such as the pancreas), or administration by any route or method that delivers a therapeutically effective amount of the drug or composition to the cells or tissue to which it is targeted. Administration can refer to introducing the nucleic acid construct to the subject as DNA or mRNA, introducing a vector containing the nucleic acid to the subject, or introducing cells that have been transduced ex vivo with a construct, such as by electroporation or using a vector to the subject.

As used herein, the terms “treatment.” “treating.” and the like, as used herein refer to obtaining a desired pharmacologic and/or physiological effect. “Treatment,” includes: (a) preventing the condition or disease or symptom thereof from occurring in a subject which may be predisposed to the condition or disease but has not yet been diagnosed as having it: (b) inhibiting the condition or disease or symptom thereof, such as, arresting its development; and (c) relieving, alleviating or ameliorating the condition or disease or symptom thereof, such as, for example, causing regression of the condition or disease or symptom thereof.

As used herein, the terms “therapeutic amount” and “effective amount” refer to an amount of the agent that results in treatment or of obtaining a desired pharmacologic and/or physiologic effect. An effective amount may be contained in one dose of the agent or may be that amount contained in a course of treatment over several or many administrations.

### 3. Summary of Results

Mesenchymal stem cell (MSC) therapy promotes limb transplant functional recovery, which is attributed to improvements in nerve regeneration, myelination, and muscle innervation.

Delivery of IL-10 via IL-10 engineered MSC enhances the MSCs' ability act as an anti-inflammatory and nerve/muscle regenerative agent in limb transplantation.

Platelet and G-CSF therapy also promote nerve regeneration and functional outcomes in the sciatic nerve transection and repair model.

### 4. Embodiments of the Invention

### A. Introduction

The invention disclosed herein relates to modified mesenchymal stem cells which are useful to improve outcomes in treatment of neuromuscular injuries, and especially in limb transplantation. For example, in the context of limb transplantation, IL10-expressing MSCs reduce inflammation and immune responses triggered by the transplanted tissue. Generally, the MSCs are isolated from a subject or patient, expanded and modified, and then reintroduced back into the patient as an autologous cell transplant. The methods also can be used for allogenic administration to a subject in some embodiments.

Methods for limb transplantation itself can be performed by any method or methods known in the art as is appropriate for the limb and/or tissue which is being transplanted. Any limb transplantation is contemplated for use with the invention, including leg, foot, arm, hand, digit or any suitable body part.

### B. MSC

In a preferred embodiment, autologous MSCs are isolated from a patient having had or planning to have an amputation of a limb and these cells then expanded in culture. In a preferred embodiment, the MSC are derived from bone marrow tissue. MSCs have an effect on macrophages, neutrophils, NK cells, mast cells and dendritic cells in innate immunity. MSCs can migrate to the site of an injury, where they may have an anti-inflammatory effect.

MSC for use in the invention are obtained as follows: bone marrow-derived MSCs were isolated and administered as previously described. Briefly, Lewis rats were euthanized and long bones (tibia, femur) harvested aseptically. Bone marrow cells (BMCs) harvested from long bones were cultured at a density of 5-10×10⁷ cells/ml in MSC complete medium. The complete medium was prepared using low glucose DMEM (Dulbecco's Modified Eagle's medium) containing Glutamax™ and pyruvate, 10% fetal bovine serum, penicillin (100 units/ml), and streptomycin [100 μg/ml] as described previously. BMCs were plated at a density of 0.5×10⁶ cells/cm² in 75 or 175 cm² flasks and cultured at 37° C. with 5% CO₂. At about 72 hours of culture, the supernatant containing non-adherent cells was removed and fresh complete medium was added. The adherent cells were further cultured until they reached about 70-80% confluence and were sub-cultured at 1:3. Ex vivo expanded MSCs (passage ≤3) were harvested and stored at −150° C. The freezing medium used was RPMI 1640 containing 10% dimethyl sulfoxide (DMSO), 30% fetal bovine serum, penicillin at 100 U/ml and streptomycin at 100 μg/ml. About one to two weeks prior to MSC injection, frozen cells were removed and expanded in cultures as described previously.

MSCs are modified to express or overexpress IL-10 as follows:

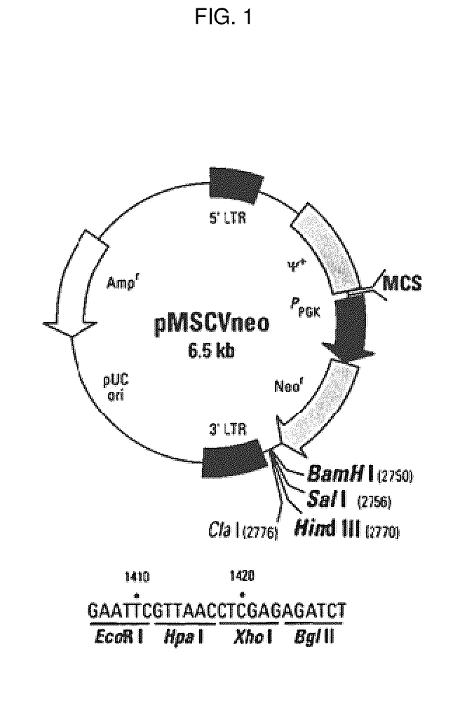

Recombinant vIL-10 (IL-10) vector construction: The recombinant vIL-10 vector was constructed as we have described previously. Briefly, vIL-10 cDNA was digested with EcoRI enzyme and the vIL-10 coding sequence (0.5 kb) was isolated and cloned into a retroviral expression vector, pMSCVneo (Clontech Laboratories, Palo Alto, CA). The clone with an intact vIL-10 gene sequence in the correct orientation (5′ to 3′) was further expanded for large-scale vIL-10-vector preparation. See FIG. 1 for the vector map used here. Two sequences are provided below. After cloning this fragment in pMSCVNeo vector, we sequenced and confirmed the sequence and orientation (5′-3′) for accuracy before transducing stem cells.

BCRF1 gene, virus coding sequence (about 0.5 kb fragment) at BCRF1 gene position 9,675-10,184.

(SEQ ID NO: 2)
atggag cgaaggttag tggtcactct gcagtgcctg gtgctgcttt
acctggcacc tgagtgtgga ggtacagacc aatgtgacaa
ttttccccaa atgttgaggg acctaagaga tgccttcagt
cgtgttaaaa cctttttcca gacaaaggac gaggtagata
accttttgct caaggagtct ctgctagagg actttaaggg
ctaccttgga tgccaggccc tgtcagaaat gatccaattc
tacctggagg aagtcatgcc acaggctgaa aaccaggacc
ctgaagccaa agaccatgtc aattctttgg gtgaaaatct
aaagacccta cggctccgcc tgcgcaggtg ccacaggttc
ctgccgtgtg agaacaagag taaagctgtg gaacagataa
aaaatgcctt taacaagctg caggaaaaag gaatttacaa
agccatgagt gaatttgaca tttttattaa ctacatagaa
gcatacatga caattaaagc cagg

Interleukin-10 BCRF1 (human gammaherpesvirus 4) amino acid sequence (170 aa).

```
(SEQ ID NO: 3)
    1 MERRLVVTLQ CLVLLYLAPE CGGTDQCDNF PQMLRDLRDA FSRVKTFFQT KDEVDNLLLK
  61 ESLLEDFKGY LGCQALSEMI QFYLEEVMPQ AENQDPEAKD HVNSLGENLK TLRLRLRRCH
121 RFLPCENKSK AVEQIKNAFN KLQEKGIYKA MSEFDIFINY IEAYMTIKAR
```

IL-10 vector production: Mammalian cell transfection was performed by the calcium phosphate method (CalPhos mammalian transfection kit; Clontech Laboratories™) as we have previously described. RetroPack PT67 (Clontech Laboratories), a fibroblast (NIH/3T3)-derived cell line designed for stable production of high-titer retrovirus, was used. Virus produced by RetroPack PT67 cells expresses a dual-tropic envelope, 10A1, that recognizes receptors on mouse, rat, human, hamster, mink, cat, dog, and monkey cells. A broad mammalian host range is caused by the fact that the virus can enter target cells via two surface molecules, the amphotropic retrovirus receptor RAM1 (Pit2) and the GALV (Pit1) receptor, such that if one receptor is not abundantly expressed by a given species or cell type, the alternative receptor may still allow viral entry). Briefly, PT67 packaging cells (1×10⁶) were plated in 25-cm² flasks 12-24 hours before transfection; when the cells became 50-80% confluent transfection was performed. One to 2 hours before transfection, 25 mM chloroquine replacement medium was exchanged to increase transfection efficiency. The calcium phosphate method (CalPhos mammalian transfection kit; Clontech Laboratories™) was used for transfection as described previously. The cells were washed twice with phosphate-buffered saline (PBS) to remove calcium phosphate and then fed with 5 ml of fresh complete growth medium and incubated at 37° C. until needed for assay. Stable transformants were selected 24-72 hours post-transfection by growing in medium containing neomycin (Geneticin 418 [G418]) at 1 mg/ml for 7 days as described previously.

Viral titer determination: The viral titer was determined as described previously. Briefly, NIH/3T3 cells (CRL-1658; American Type Culture Collection [ATCC], Manassas, VA) were plated in 6-well plates (10⁵ cells per well). After 12-24 hours, One-ml aliquots of 10-fold serial dilutions of the viral supernatant (collected from the virus-packaging PT67 cells) were added to each well. After 48 hours, the cells were selected by culturing for 1 week in medium containing antibiotic G418 (1 mg/ml). Viral titers corresponded to the number of colonies present at the highest dilution (number of colonies×the dilution). The viral supernatants had a titer of 5×10⁶ particles/ml and were used to transduce MSCs.

MSC engineering: MSC engineering with vIL-10 was performed similar to the protocol described previously for hematopoietic stem cell. Briefly, MSCs isolated from rat bone marrow and expanded ex vivo (passages 3 to 6) were plated (10⁶ cells per 25-cm² plate) and cultured in complete medium. After 24-48 hours of culture, 75% of the medium was replaced by adding viral supernatants derived from vIL-10 vector or vector DNA-engineered PT67 cell cultures. Infection was conducted for 6 hours in the presence of Polybrene™ (8 mg/ml of medium) and the supernatant was removed and replaced with fresh complete medium. The transduction was repeated for the second time on the next day in the same manner. After transduction (48-72 hours), the supernatants were assayed for vIL-10 production by enzyme-linked immunosorbent assay (ELISA) to confirm the transduction of MSCs. A human IL-10 detection kit (555157; BD Biosciences™), which recognizes vIL-10, was used as per the manufacturer's recommendations. Stable transfectants were selected by culturing transduced cells in G418 (1 mg/ml) for 7 days. MSCs that produced IL-10 ex vivo are injected into animals.

C. IL-10 engineered MSCs
Ex Vivo vIL-10 Production:

Ex vivo-expanded MSCs were efficiently transduced with 75% rvIL-10 retrovirus supernatants and incubated for 6 hours (transduction was repeated at 24 hours). The transduced cells (MSC-vIL10) produced about 4 ng of vIL-10 per 10⁶ cells in 24-hour culture supernatants, as measured by ELISA. The engineered MSC survival in G418 medium, and expression of vIL-10, demonstrated successful gene transduction.

In Vivo vIL-10 Production:

After MSC-vIL-10 administration, intra lung vIL-10 mRNA message was detectable by RT-PCR analysis in the vIL-10-preconditioned animals. As expected, no vIL-10 mRNA expression was detected in the control animals administered empty vector-MSCs or in reverse transcriptase negative PCR controls.

### Lung Function was Significantly Improved in MSC-vIL10-Treated Animals:

As early as 4 hours after IR injury mean arterial blood oxygenation (PaO2=FiO2 [P:F] ratio, mmHg) was significantly (p<0.05) higher in the MSC-vIL-10 group (319±95; n=7) compared with the untreated saline group (63±19; n=6) or MSC-empty vector control group. At 24 hours post-IR injury the mean P:F ratio was higher (p<0.05) in the MSC-vIL10 group (353±105; n=10) compared with the MSC-empty vector group (138±85; n=9) and saline group (88±39; n=10). By days 3 and 7 the P:F ratios approached normal in the MSC-vIL-10 group (475±55; n=10 and 434±33; n=9), but not in the MSC-empty vector (238±165; n=10 and 217±158; n=11) or saline (198±141; n=9; and 187±149) control groups. In sham controls, the base P:F ratio was 482±24 mm.

### Lung Histopathology Demonstrated Reduced IR Injury Score in MSC-vIL10-Treated Animals:

After ischemia-reperfusion, lungs were harvested at 4 hours, 24 hours, 72 hours and 7 days, and analyzed for histopathology by examining hematoxylin and eosin (H&E)-stained sections to determine injury scores. The baseline mean injury score was significantly (p<0.05) higher at 4 hours (2.0±1.4; n=5), compared with IR injury scores of lungs harvested at later time points, that is, 24 hours (1.1±0.7; n=5), 72 hours (0.5±0.5; n=5), and 7 days (0.8±0.3; n=4). In sham control animals with no IR injury the score was 0.2±0 (n=5). At 4 hours after IR injury, the MSC-vIL10 group had a significantly (p<0.05) lower injury score (0.9±0.4; n=7) compared with the no treatment control group (2.5±1.4; n=6; see FIG. *** 4). However, injury scores at 24 hours, 72 hours, and 7 days were not significantly different (p<0.05) between the MSC-vIL10-treated and untreated groups.

### IL-10 Delivery Via Engineered Hematopoietic Stem Cell (HSC) Significantly Prolonged Cardiac Allograft Survival:

Lethally irradiated (9.5 Gy) CBA/J mice were reconstituted with syngeneic vIL-10-HSC, vector-DNA-HSC or unengineered (UE) HSC (4×10⁶) administration at least 6 weeks prior to allogeneic (C57BL/6) heart transplantation. Cardiac allograft survival in animals that received UE-HSC or vector-DNA-HSC, and vIL-10-HSC was 16±3 (n=6) and 71±40 (n=8) days, respectively. In nonirradiated animals that did not receive any HSC administration, allograft survived for 11±1 (n=5) days. Cardiac allograft survival was significantly prolonged (p) <0.004) in animals that received vIL-10-HSC compared to the control groups that received no HSC, UE-HSC, or vector-DNA HSC. Sublethally irradiated (4 Gy) CBA/J mice were given vIL-10-HSC, vector-DNA-HSC, or unengineered HSC (6×10⁶) at least 6 weeks prior to allogeneic (C57BL/6) heart transplantation. Cardiac allograft survival in animals that received UE-HSC or vector-DNA-HSC, and vIL-10-HSC was 12±1.0 (n=6) and 114±15 (n=3) days, respectively. Cardiac allograft survival was significantly prolonged (p<0.002) in the animals that received vIL-10-HSC compared to the control groups that received UE-HSC or vector-DNA-HSC. These results demonstrate IL10's potential to mitigate inflammation and prolong graft survival.

### D. Pharmaceutical Compositions

The modified MSCs are formulated into a composition suitable for administration to the patient. Preferably, MSCs are formulated for intravenous injection and local injection into the site to be treated, however, any suitable formulations known in the art for administration to a patient can be used.

In preferred embodiments, the MSC compounds described herein are formulated and are administered as a pharmaceutical composition that includes a pharmaceutically acceptable carrier and one or more pharmaceutical agent, including one or more of the inventive compounds described herein, and optionally including one or more of the inventive compounds described herein in combination with an additional agent. A pharmaceutically acceptable carrier refers to any convenient compound or group of compounds that is not toxic and that does not destroy or significantly diminish the pharmacological activity of the therapeutic agent with which it is formulated. Such pharmaceutically acceptable carriers or vehicles encompass any of the standard pharmaceutically accepted solid, liquid, or gaseous carriers known in the art, such as those discussed in the art.

A suitable pharmaceutical carrier depends on the route of administration contemplated for the pharmaceutical composition. Routes of administration are determined by the person of skill according to convenience, the health and condition of the subject to be treated, and the location and stage of the condition to be treated. Such routes can be any route which the practitioner deems to be most effective or convenient. For example, routes of administration can include, but are not limited to local or parenteral. For example, the following routes of administration are contemplated as useful for the invention described herein, including intravenous, intraarterial, intrathecal, subcutaneous, intraperitoneal, local injection to the site of tissue damage or transplanted tissue, direct injection into a transplanted limb or blood vessels that supply the transplanted limb, and the like. The administration can be given by transfusion or infusion, and can be administered by an implant, an implanted pump, or an external pump, or any device known in the art. Preferably, the MSC according to this invention are administered by intravenous injection, local injection, or both.

Therefore, the forms which the pharmaceutical composition can take generally include but are not limited to: sterile solutions or other liquids for injection or infusion, suspensions, emulsions, lipid vesicles, hydrogels (such as Hyaluronan-Heparin based pre-made hydrogel, ESI Bio. Inc.™. USA), and the like. Preferred dosage forms are hydrogels and sterile suspensions, solutions, or emulsions for injection.

Treatment regimens include a single administration or a course of administrations lasting two or more days, including a week, two weeks, several weeks, a month, two months, several months, a year, or more, including administration for the remainder of the subject's life. The regimen can include multiple doses per day, one dose per day or per week, for example, or a long infusion administration lasting for an hour, multiple hours, a full day, or longer. A preferred dosage regimen in certain embodiments includes a local injection and an intravenous injection immediately after the transplant is performed and further doses at regular intervals of intravenous injection or infusion over a period of several days or weeks, or longer.

Dosage amounts per administration include any amount determined by the practitioner, and will depend on the size of the subject to be treated, the state of the health of the subject, the route of administration, the severity of the condition to be treated or prevented, and the like. In general, it is contemplated that for the majority of human subjects, a dose in the range of about 1-10×10⁶/kg body weight would be sufficient. This dose can be administered locally or systemically weekly, daily, or multiple times per day as needed.

The invention can be used with agents or other methods that act as nerve and muscle regenerative agents, for example concomitant treatment that includes shockwave treatment, administration of G-CSF. platelet-rich plasma, or a combination thereof as nerve and muscle regenerative agents to the limb transplant or peripheral nerve injury recipient.

### 5. Examples

This invention is not limited to the particular processes, compositions, or methodologies described, as these may vary. The terminology used in the description is for the purpose of describing the particular versions or embodiments only, and is not intended to limit the scope of the present invention which will be limited only by the appended claims. Although any methods and materials similar or equivalent to those described herein can be used in the practice or testing of embodiments of the present invention, the preferred methods, devices, and materials are now described. All publications mentioned herein, are incorporated by reference in their entirety: nothing herein is to be construed as an admission that the invention is not entitled to antedate such disclosure by virtue of prior invention.

### Example 1: General Methods

### A. Animal Models

### Limb Transplantation Model:

The general surgical techniques used for orthotopic limb transplantation model are as previously described. Briefly, the animal was anesthetized with intraperitoneal (IP) injection of ketamine (40-80 mg/kg) and xylazine (5-10 mg/kg), and anesthesia was maintained with inhalant 1-2% isoflurane. The surgical site was prepared, and a skin incision was made around the circumference of the right hind limb at the level of the inguinal ligament. Beginning laterally, the skin of the upper thigh was mobilized to expose the biceps femoris. The biceps femoris was then divided near the distal attachments to the stifle and tibia, leaving a sufficient edge of residual tissue for later repair. The biceps was reflected to expose the sciatic nerve within the stifle fossa. The sciatic nerve was dissected out proximally to the point of emergence from below the gluteus muscle, preserving mesoneurial tissue. The sciatic nerve was transected proximally after a tag suture of 10-0 nylon was placed. The femoral vessels were isolated from the inguinal ligament to the level of the epigastric takeoff. Tag sutures of 10-0 nylon were placed proximally on the femoral vessels. All muscle groups were then sharply divided slightly proximal to the level of the mid-femur. Heparin (300 mL (50 IU)) was administered via the tail vein for anticoagulation. The femoral vessels were then clamped above the previously placed suture tags and transected. Using a 22-gauge angiocatheter, approximately 5-10 mL of ice-cold Plegisol™ solution (Hospira™, Lake Forest, IL) was perfused through the femoral artery, until the venous effluent was clear. The osteotomy was performed at the mid femur using a rotary saw (DREMELVR 7300-N/8 Mini-Mite 4.8-V, Robert Bosch Tool Corporation™, Racine, WI) with a stainless-steel saw blade. Once detached, the donor limb (graft) was wrapped in moist gauze and placed on ice, until the recipient animal was prepared and ready for transplantation. The donor animal was euthanized.

The recipient animal received a preoperative antibiotic, cefazolin (25 mg/kg body weight) subcutaneously (SQ) and was anesthetized and prepared for surgery. The right hind limb was removed in the same fashion as for the donor, with division of the femoral vessels, sciatic nerve, and muscle groups occurring further distally to preserve adequate tissue for approximation with the donor limb. The donor limb was transplanted by performing osteosynthesis of the femur using an intramedullary pin, as described previously and orthopedic bone cement as known in the art to achieve a rigid fixation. The vascular anastomoses were performed using the vascular cuff technique, as described previously. Briefly, thin-walled polyimide tubing (Rivertech Medical LLC™, Chattanooga, TN) was used to fashion arterial (0.724 mm inside diameter, 0.025 mm wall thickness) and venous (1.151 mm inside diameter, 0.025 mm wall thickness) cuffs approximately 2.5 mm in length. The sciatic nerve was repaired using 2-4 simple interrupted epineurial sutures of 10-0 nylon. MSCs or vehicle were administered topically at the sites of the nerve repair and vascular anastomoses and injected at the site of muscle repair and bone fusion. All muscle groups were approximated with 6-0 prolene. The skin was closed with interrupted 4-0 nylon sutures and stainless-steel clips.

### Sciatic Nerve Repair Model:

Sciatic nerve repair model is an alternate model to study the efficacy of MSC reagents in peripheral nerve injury and repair that we have described previously. This model is less intensive to perform and focuses on nerve injury and muscle innervation. Briefly, the rat was anesthetized with intraperitoneal injections of ketamine (40-80 mg/kg b.wt.) and xylazine (5-10 mg/kg b.wt.); we used inhalant 1-2% isoflurane to maintain anesthesia. Rats were treated with cefazolin (25 mg/kg b.wt. SQ) a preoperative antibiotic, and Vidisic™ (ophthalmic ointment) was applied to the eyes to prevent corneal drying. We prepared the surgical site by clipping hair and sterilizing with 10% chlorhexidine and 70% alcohol. The animal was placed on a thermos regulated warming pad to maintain body temperature at about 38° C.

A circumferential skin incision around the right mid-thigh region was made. The biceps femoris was exposed and divided near the distal attachments to the stifle and tibia; the muscle was then reflected to expose the sciatic nerve. The sciatic nerve dissected out proximally to the point of emergence from below the gluteus muscle. Tag sutures of 10-0 nylon were placed on proximal and distal ends of the sciatic nerve, and the nerve was transected proximal to the trifurcation of sciatic nerve into tibial, peroneal, and sural nerves as described previously. For anti-coagulation, 300 μl (50 U) heparin was administered via the tail vein. The animal then was monitored under anesthesia for an hour without any further manipulation to mimic a traumatic peripheral nerve injury/damage situation with transport time to a facility for treatment. Neurorrhaphy (sciatic nerve proximal and distal end approximation) was performed with 10-0 nylon sutures, followed by biceps femoris repair and skin closure.

### B. Cells and Cell Culture

Cell isolation and culturing will be done as described previously. Bone marrow cells (BMCs) were isolated from the bones and suspended at 5-10×10⁷ cells/ml in MSC complete or growth medium. The complete medium was prepared using Dulbecco's Modified Eagle's medium (DMEM)-Low glucose, Glutamax™, pyruvate, 10% fetal bovine serum, penicillin (100 units/ml), and streptomycin (100 mg/ml); all reagents were obtained from Gibco/Life Technologies™, NY. Isolated BMCs were plated (0.5×10⁶ cells/cm²) in 75 or 175 cm² flasks and cultured at 37° C. in 5% CO₂ in complete medium. About 72 hours following culture, non-adherent cells (floating) in the supernatant were removed completely and the medium was replaced with fresh complete medium. Adherent cells were cultured for an additional 2-4 weeks; when the cultures were about 70% confluent, they were sub-cultured (1:3). Early passage (≤3) MSCs were harvested and frozen in RPMI 1640 medium containing 10% dimethyl sulfoxide (DMSO), 30% fetal bovine serum, penicillin (100 U/ml) and streptomycin (100 μg/ml). The cells were stored at −150° C. for future use. About one to two weeks prior to MSC injection, frozen cells were removed, quickly thawed in a water bath (37° C.) and expanded in cultures using complete medium. Expanded cells were harvested, counted and administered to animals.

### C. Pharmaceutical Compositions

MSCs (2×10⁶/rat) suspended in about 0.4 ml of 1× hydrogel (Hyaluronan-Heparin based pre-made hydrogel, ESI Bio. Inc.™, USA) and administered locally at the transplant site. Immediately after surgery, 1.0-1.5 ml of MSCs (2×10⁶/rat in saline) or saline (vehicle control) was injected intravenously (IV) via the dorsal penile vein. Similar formulation and dose (2×10⁶/kg b.wt.) appears to be good for human use.

### Example 2: Orthotopic Syngeneic Hind-Limb Transplant in Lewis (RT1.A¹) Rats

To study promotion of nerve regeneration and functional outcomes in limb transplantation by bone marrow-derived mesenchymal stem cell therapy, Lewis rats were subjected to hind-limb amputation and transplantation. Following the transplant, the rats received syngeneic MSC (2×10⁶; passage ≤6) or vehicle (saline control), by injection, intravenously and locally using a hydrogel formulation.

Limb sensory function (SF) and motor function (MF) were assessed by cutaneous pain reaction test and walking track analysis, respectively. At study endpoint (16 weeks) limb tissues were harvested for cell and molecular analysis.

The limb SF in the tibial, peroneal/sural, and saphenous nerve boundaries recovered by 5, 6 and 8 weeks, respectively. Total SF in the limb did not vary significantly (P>0.05) between MSC-treated and untreated controls. Limb SF recovery was gradual. See Table 1 for results graded on a scale of 0-3, where 0 indicates no function and 3 indicates optimal function.

TABLE 1
Sensory Function Recovery Results

| Week 8 | Week 12 | Week 16 |
|---|---|---|
| 1.3 | 2.3 | 2.6 |

Walking track analysis did not produce clear footprints to calculate a Sciatic Function Index (SFI), so a unique grading system on a scale of Grade 0-4 was developed to assess MF. MF in the transplanted limbs was significantly improved (P<0.05) with MSC treatment compared to control at week 8 and at week 16. See Table 2, below.

TABLE 2
Motor Function Recovery Results.

| | |
|---|---|
| Week 8 | Week 16 |

| MSC Treatment | Control | MSC Treatment | Control |
|---|---|---|---|
| 1.3 | 0.5 | 2.6 | 0.6 |

Ex vivo expanded Rat MSCs were CD29+, CD31−, CD34−, CD44+, CD45low, CD90+, MHC Class I+, Class II- and pluripotent. Neurofilament M (axonal marker), S100 and Krox20 (myelinating Schwann cells), Oct6 (Pre-myelinating Schwann Cells) and GAP43 (regeneration marker) expressions were significantly (P<0.05) increased and GFAP (non-myelinating Schwann cells) was reduced (P<0.05) in the distal portion of the sciatic nerve in MSC treated rats compared to control. Gastrocnemius muscle atrophy was significant (P<0.05) and animals developed flexion-contractures in the transplanted limb; however, contractures were mild in MSC-treated (Grade 0.9) compared to control (Grade 1.6) rats on a scale of 0-3 (0, No contracture; 3, severe contracture) at study endpoint.

Thus, MSC therapy appears to promote limb transplant functional recovery and is attributed to enhanced nerve regeneration, myelination, and muscle innervation.

### REFERENCES

All publications listed below and throughout the specification are hereby incorporated by reference in their entirety. Nothing herein is to be construed as an admission that the invention is not entitled to antedate such disclosure by virtue of prior invention.
1. Fitzpatrick E B, Dehart M J, Brown T A, Salgar S K. Mesenchymal stem cell therapy to promote limb transplant functional recovery. Microsurgery. March 2017; 37(3):222-234. doi:10.1002/micr.30068
2. Weiss J B, Phillips C J, Malin E W, Gorantla V S, Harding J W, Salgar S K. Stem cell, Granulocyte-Colony Stimulating Factor and/or Dihexa to promote limb function recovery in a rat sciatic nerve damage-repair model: Experimental animal studies. Ann Med Surg (Lond). November 2021; 71:102917. doi:10.1016/j.amsu.2021.102917
3. Bingham J R, Kniery K R, Jorstad N L, Horkayne-Szakaly I, Hoffer Z S, Salgar S K. “Stem cell therapy to promote limb function recovery in peripheral nerve damage in a rat model”—Experimental research. Ann Med Surg (Lond). May 2019; 41:20-28. doi:10.1016/j.amsu.2019.03.009
4. Salgar S K, Yang D, Ruiz P, Miller J, Tzakis A G. Viral interleukin-10-engineered autologous hematopoietic stem cell therapy: a novel gene therapy approach to prevent graft rejection. Hum Gene Ther. February 2004; 15 (2): 131-44. doi:10.1089/104303404772679940
5. Manning E, Pham S, Li S, Vazquez-Padron R, Mathew J, Ruiz P, and Salgar S K. Interleukin-10 delivery via mesenchymal stem cells: a novel gene therapy approach to prevent lung ischemia-reperfusion injury. Hum Gene Ther. June 2010; 21 (6): 713-27. doi:10.1089/hum.2009.147
6. Vu M, Bingham J, Gorantla V and Salgar S K. Mesenchymal stem cell therapy to promote functional recovery in a limb transplant model. J Immunol, 2023:210 (1 Supplement): 173.26.

What is claimed is:

1. A method of improving nerve and muscle regeneration and functional outcome for limb transplantation and peripheral nerve injury in a mammalian limb transplant or peripheral nerve injury recipient in need thereof, comprising administering to the limb transplant or peripheral nerve injury recipient a therapeutic amount of mesenchymal stem cells.

2. The method of claim 1 wherein the mammalian limb transplant recipient or peripheral nerve injury is a human.

3. The method of claim 1 wherein the limb is selected from a digit, a hand, an arm, a foot, and a leg and peripheral nerve injuries of that region.

4. The method of claim 1 wherein the mesenchymal stem cells are genetically modified to express interleukin-10.

5. The method of claim 1 wherein inflammation is reduced and tissue regeneration promoted in the transplanted limb or peripheral nerve injury region.

6. The method of claim 1 wherein graft versus host disease is reduced in the limb transplant recipient.

7. The method of claim 1 wherein the functional outcome of the limb transplant or peripheral nerve injury is improved.

8. The method of claim 1, further comprising administering concurrent or sequential shock wave therapy to the limb transplant recipient or peripheral nerve injury recipient.

9. The method of claim 1, further comprising administering G-CSF, platelet-rich plasma and shockwave therapy, or a combination thereof as nerve and muscle regenerative agents to the limb transplant or peripheral nerve injury recipient.

10. A genetically modified mesenchymal stem cell that expresses interleukin-10.

11. A pharmaceutical composition comprising a pharmaceutically acceptable carrier and the genetically modified mesenchymal stem cell of claim 10.


SEQUENCE LISTING (3 sequences)

SEQ ID NO: 1 - DNA, 24 bp, synthetic construct
  source 1..24: mol_type = other DNA
gaattcgttaacctcgagagatct

SEQ ID NO: 2 - DNA, 510 bp, synthetic construct
  source 1..510: mol_type = other DNA
atggagcgaaggttagtggtcactctgcagtgcctggtgctgctttacctggcacctgagtgtggaggtacagaccaatgtgacaattttccccaaatgttgagggacctaagagatgccttcagtcgtgttaaaacctttttccagacaaaggacgaggtagataaccttttgctcaaggagtctctgctagaggactttaagggctaccttggatgccaggccctgtcagaaatgatccaattctacctggaggaagtcatgccacaggctgaaaaccaggaccctgaagccaaagaccatgtcaattctttgggtgaaaatctaaagaccctacggctccgcctgcgcaggtgccacaggttcctgccgtgtgagaacaagagtaaagctgtggaacagataaaaaatgcctttaacaagctgcaggaaaaaggaatttacaaagccatgagtgaatttgacatttttattaactacatagaagcatacatgacaattaaagccagg

SEQ ID NO: 3 - protein, 170 aa, Homo sapiens
MERRLVVTLQCLVLLYLAPECGGTDQCDNFPQMLRDLRDAFSRVKTFFQTKDEVDNLLLKESLLEDFKGYLGCQALSEMIQFYLEEVMPQAENQDPEAKDHVNSLGENLKTLRLRLRRCHRFLPCENKSKAVEQIKNAFNKLQEKGIYKAMSEFDIFINYIEAYMTIKAR